(19) United States
(12) Patent Application Publication
(10) Pub. No.: US 2015/0313748 A1
(43) Pub. Date: Nov. 5, 2015

(54) CONFIGURABLE PERSONAL THERAPY DEVICE

(71) Applicants: Jonrow Kazzaz, Walnut Creek, CA (US); Munira Jamil, Walnut Creek, CA (US)

(72) Inventors: Jonrow Kazzaz, Walnut Creek, CA (US); Munira Jamil, Walnut Creek, CA (US)

(21) Appl. No.: 14/638,941

(22) Filed: Mar. 4, 2015

Related U.S. Application Data
(60) Provisional application No. 61/988,165, filed on May 3, 2014.

(51) Int. Cl. A61F 5/37; A63B 23/16

(52) U.S. Cl. CPC A61F 5/37; A63B 23/16

(57) ABSTRACT

A therapy device comprising is disclosed. One embodiment of the therapy device includes a casing comprising a flexible material, one or more individual units encased within the casing, comprising substantially spherical objects having a diameter in a range of 2.5 centimeters to 18 centimeters, wherein between each individual unit there is an inter-unit region that is variable in size. A loop fastener fixed to a terminal of one of the individual units allows a user to loop the device around ins or her neck.

### BACKGROUND

There exists a large demand among travelers, physical therapists, students, large music or sporting event attendees, and those who work at desks or in sitting positions for comfortable and customizable neck, head and body padding.

The current solutions for personal therapy devices such as headrests, heating pads or neck braces typically serve one purpose only and are not configurable, or even portable in some instances. Some users prefer the ability to have a very comfortable neck support that can be manipulated to hold their chin up for example. Others desire a solution that is customizable so that it is not limited to the application of the neck or head, but rather can be used to support other parts of the body. Current solutions are also not easy to store. Those that are easy to store are likely inflatable, which do not provide the same kind of comfort, and support that filled or padded support or braces do. Current solutions, even if they are very comfortable for their application still are limited because they do not offer the ability of the user to shape it to their preference.

### BRIEF DESCRIPTION OF THE DRAWINGS

FIG. 1A is a plan view of a configurable personal therapy device according to an embodiment

FIG. 1B is a cross sectional diagram of a configurable personal therapy device according to an embodiment.

FIG. 1C is a cross sectional diagram of one unit of a configurable personal therapy device according to an embodiment.

FIG. 1D is a cross sectional diagram of one unit of a configurable personal therapy device according to an embodiment.

FIG. 2 is a diagram illustrating one method for configuring a personal therapy device according to an embodiment.

FIG. 3 is a diagram illustrating one method for configuring a personal therapy device according to an embodiment.

FIG. 4 is a diagram of one use case of the personal therapy device.

FIG. 5 is a diagram of one use case of the personal therapy device.

FIG. 6 is a diagram illustrating one method for configuring a personal therapy device according to an embodiment.

FIG. 7 is a diagram of an embodiment of a personal therapy device.

FIG. 8 is a diagram of one use case of the personal therapy device of FIG. 7.

### DETAILED DESCRIPTION

Embodiments of the invention include a support device that can be manipulated to support a person's head, neck, or other body part during sitting, standing, laying or prone positions. The shape and size of the support device is adjustable to support a person's head, neck, legs, back or other body part.

Embodiments of the invention include several spherical or other shaped units made of foam, rubber or other synthetic materials designed to absorb impact and provide support when shaped. These units in some embodiments are whole or filled and in other embodiments are hollow. These units are in a casing made with nylon, spandex, polyester or other materials that allow for the configuration of the shape and retention of the desired form of the therapy device, In one embodiment of the invention the casing of the units creates ridges that allow the therapy device to have a massage effect when tapped, rubbed or slid against a user's body. In one embodiment the comfort of the user is taken into consideration as this fabric or synthetic material acting as the casing is soft and comfortable to the touch. In another embodiment, the shape of the therapy device would be like a collar or horse-shoe, with an opening for the therapy device to be placed around the body part or user for support. In another embodiment, the natural shape of the therapy device is straight or only slightly curved, not a rigid horseshoe shape.

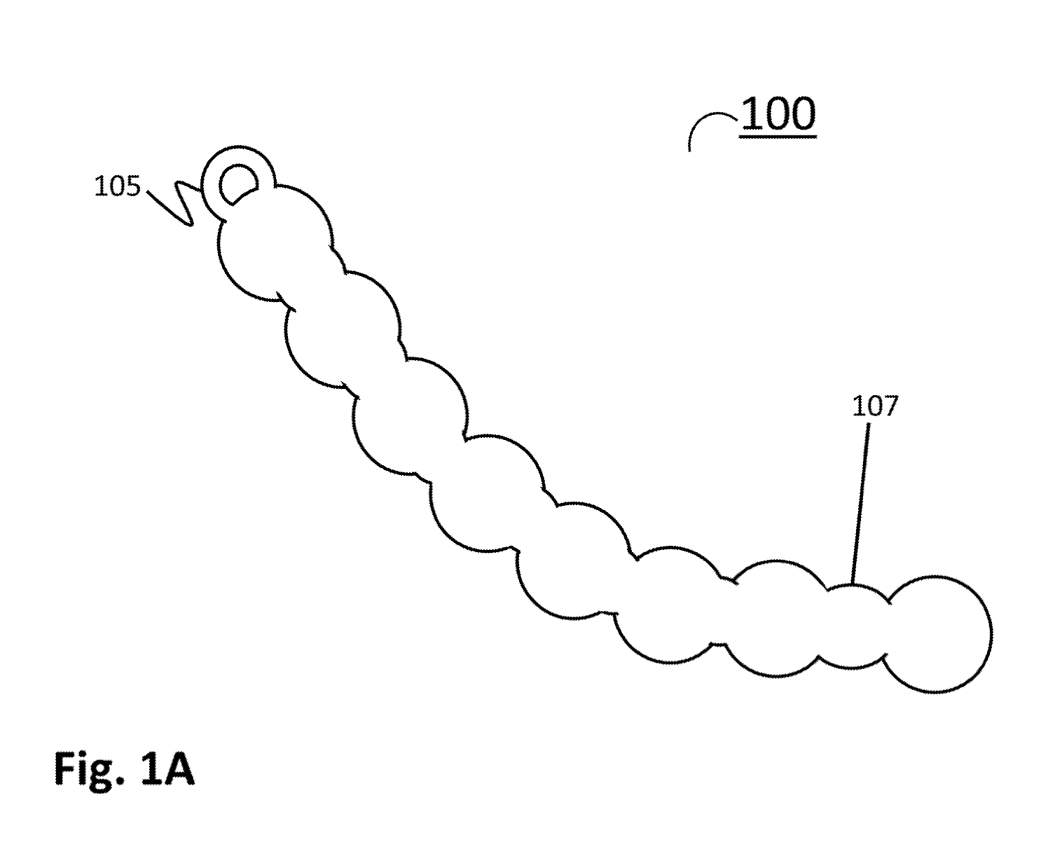

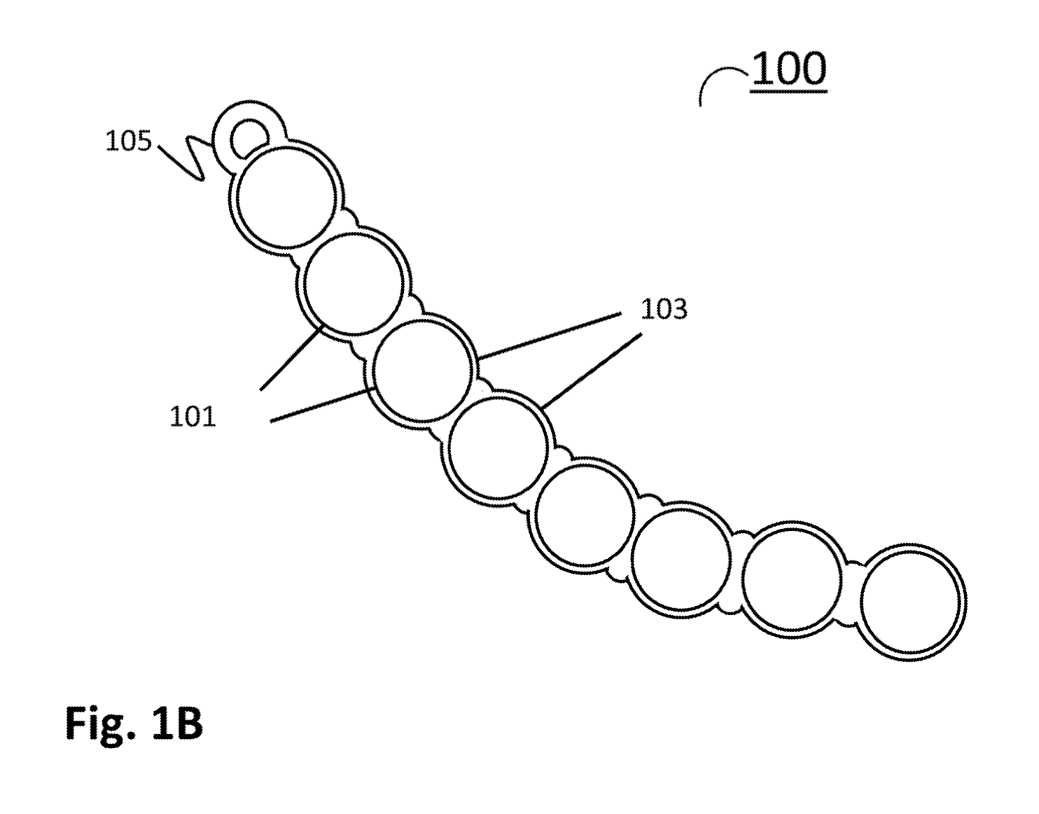

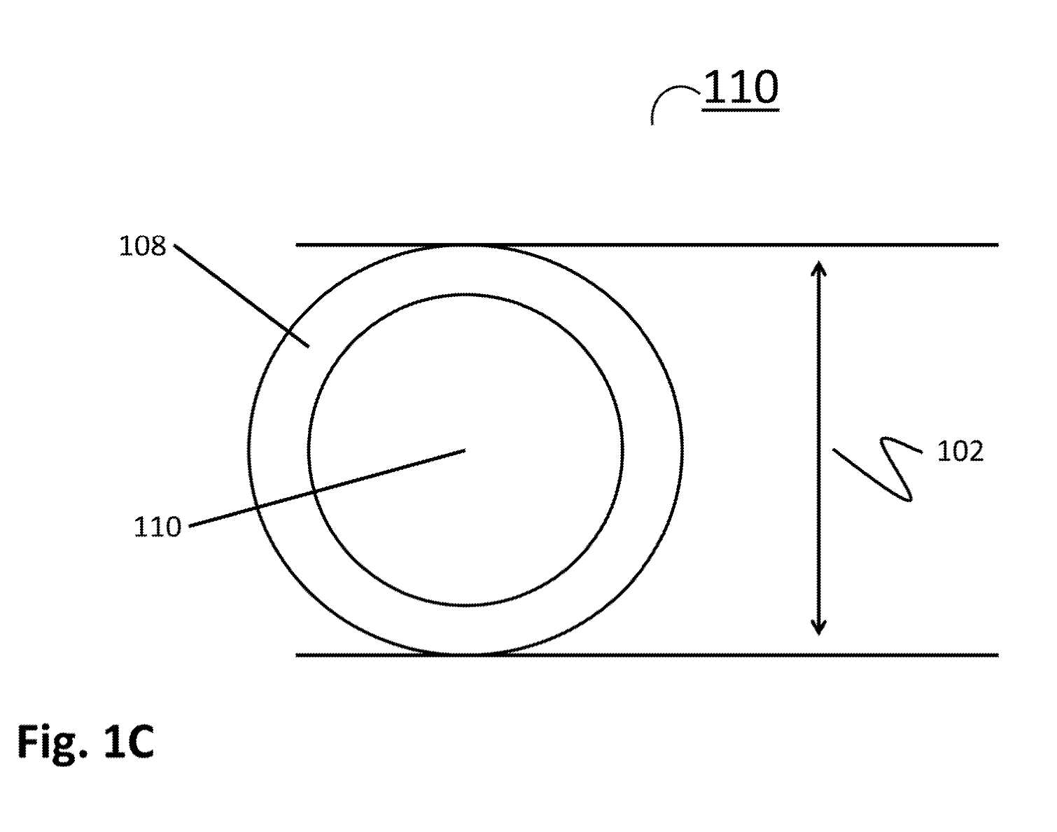

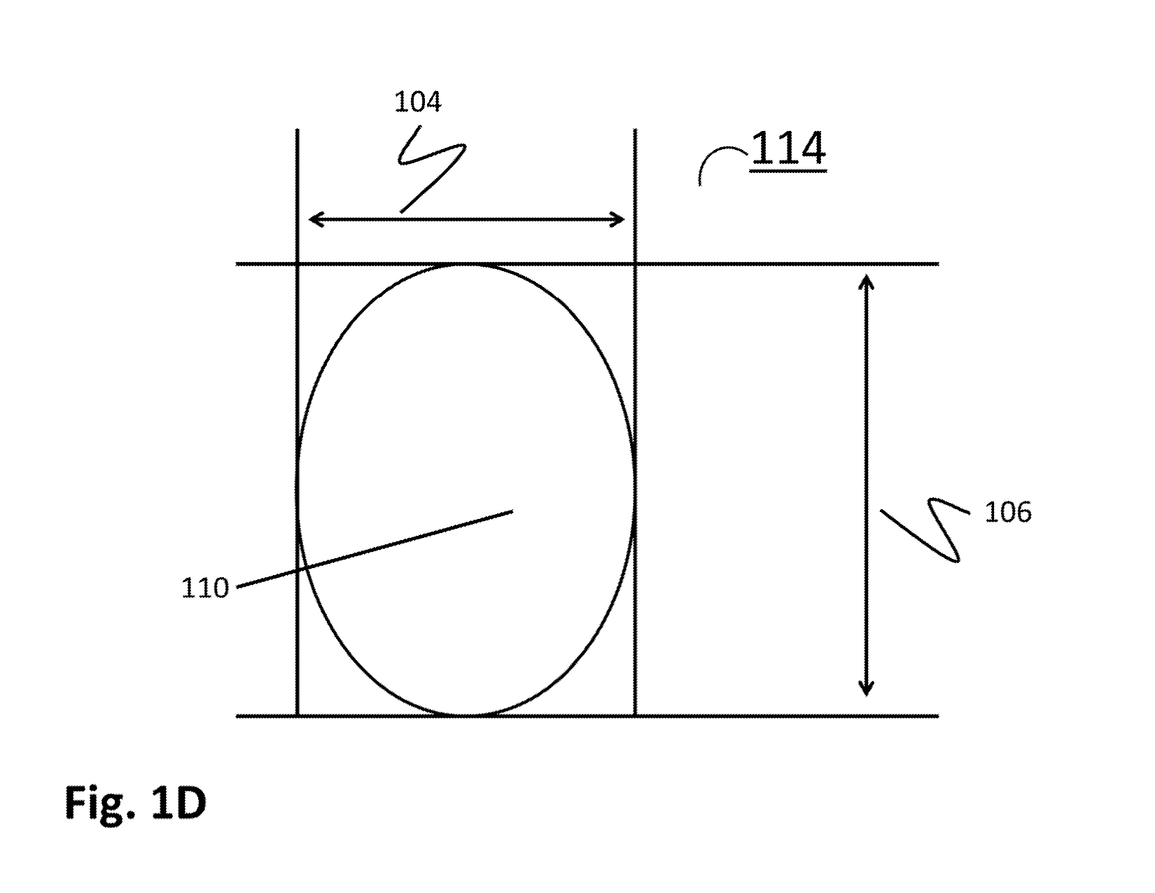

FIG. 1A-1E shows an embodiment of the therapy device, FIG. 1A shows the therapy device in a natural resting position with a slight curve to its shape. FIG. 1B shows a cross section of an embodiment of the therapy device. The therapy device contains individual units 101. The individual units 101 are wrapped tightly in the casing 103. At the top of the support padding is a loop fastener 105 made of the same fabric as the casing 103. The loop fastener 105 can be used to hang the therapy device from a hook or for configuration. The inter-unit region 107 is an area between two units that is of varying size depending on the distance between the two units. FIG. 1C shows an embodiment of a cross section of a unit 101, in this embodiment the unit 101 is hollow. The wall 108 of the unit 101 can be rigid or soft. The wall 108 can be compressed. The wall 108 can be fabric as in the example of being filled with beads, or alternatively, cavity 110 can be empty and wall 108 can be different width or dimension to accommodate different degrees of deformability. In one example, wall 108 will range in thickness between 0.2 centimeter and 1 centimeter. The dimension of 108 is not meant to be limiting, any feasible dimension is possible. The cavity 110 can be filled with bead like objects, such as polystyrene beads, that give the unit 101 more weight or support. The dimension 102 of unit 101 can be different to accommodate various applications for the therapy device. In one example, diameter 102 could range from 2.5 centimeters to 14 centimeters. The diameter 102 is not meant to be limiting, any feasible dimension is possible. FIG. 1D shows a cross section of unit 114 that would be contained in an embodiment of the therapy device. In this embodiment the width 104 and height 106 of unit 114 are not equal. The shape of the unit 114 can be oblong or uneven for different embodiments of the therapy device. That is, although the unit can be substantially spherical or symmetrical in all dimensions, the unit can also not be symmetrical in all dimensions.

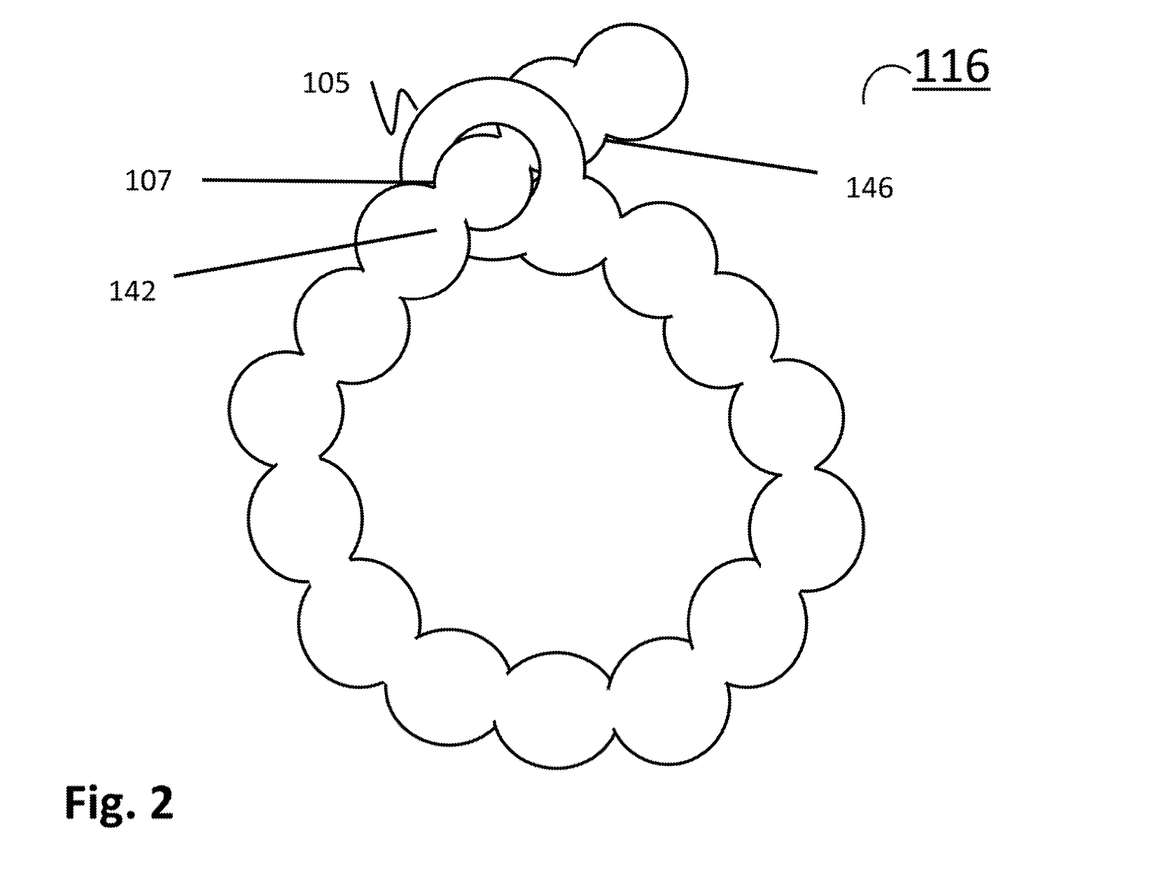

FIG. 2 shows the configuration of the therapy device according to an embodiment. The loop fastener 105 can be used to configure the therapy device into a fixed position or loop by slipping one or more of the units 101 through the loop fastener 105. The loop fastener 105 being made of a flexible material allows for the fastener to expand and then contract around the inter-unit region 107 between the units 140 and 142. The loop fastener 105 can also be used to affix the therapy device to a piece of furniture or a user's body part for added configurability.

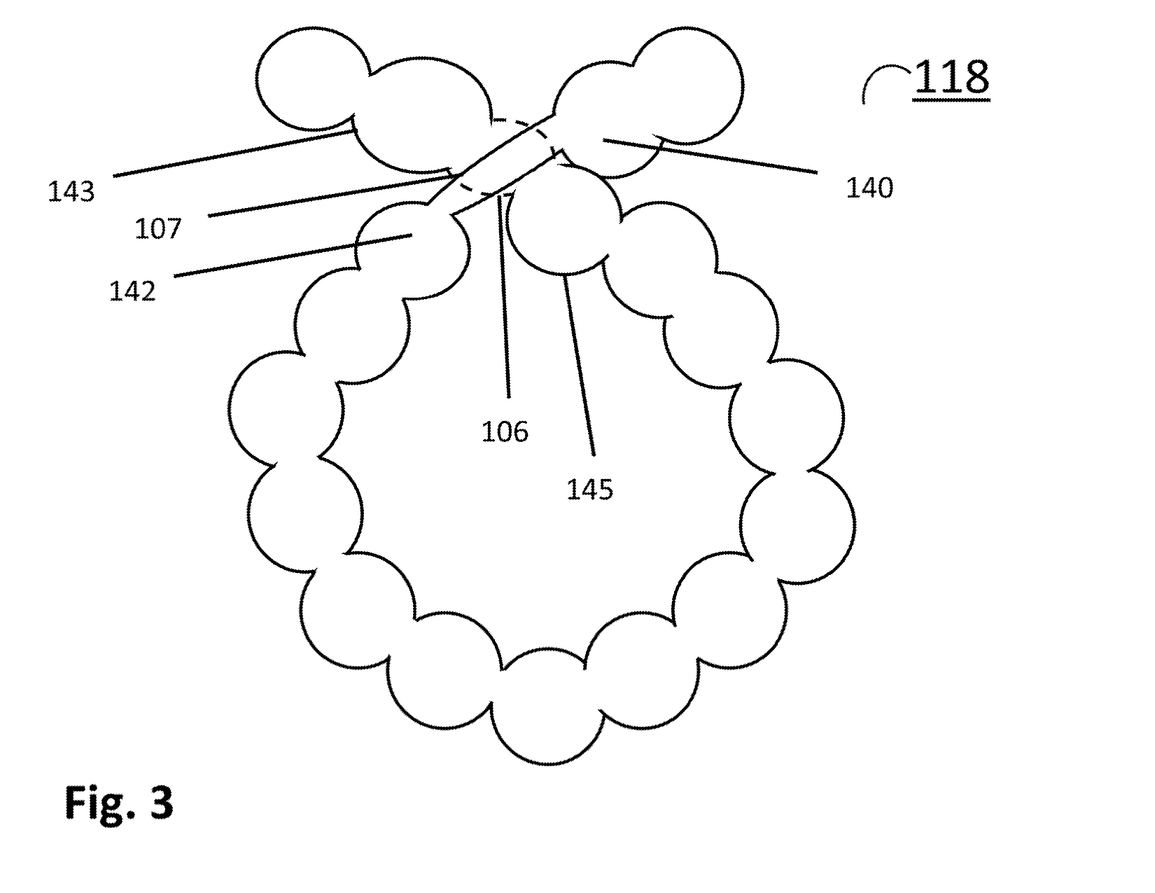

FIG. 3 shows the configuration of the therapy device according to an embodiment. The therapy device 118 is shown in its configuration as a loop. The configuration is achieved by placing inter-unit region 106 over inter-region unit 107 and twisting to create tension. The inter-region unit 107 is laid over inter-region unit 106 and unit 140 and 142 are twisted around units 143 and 145.

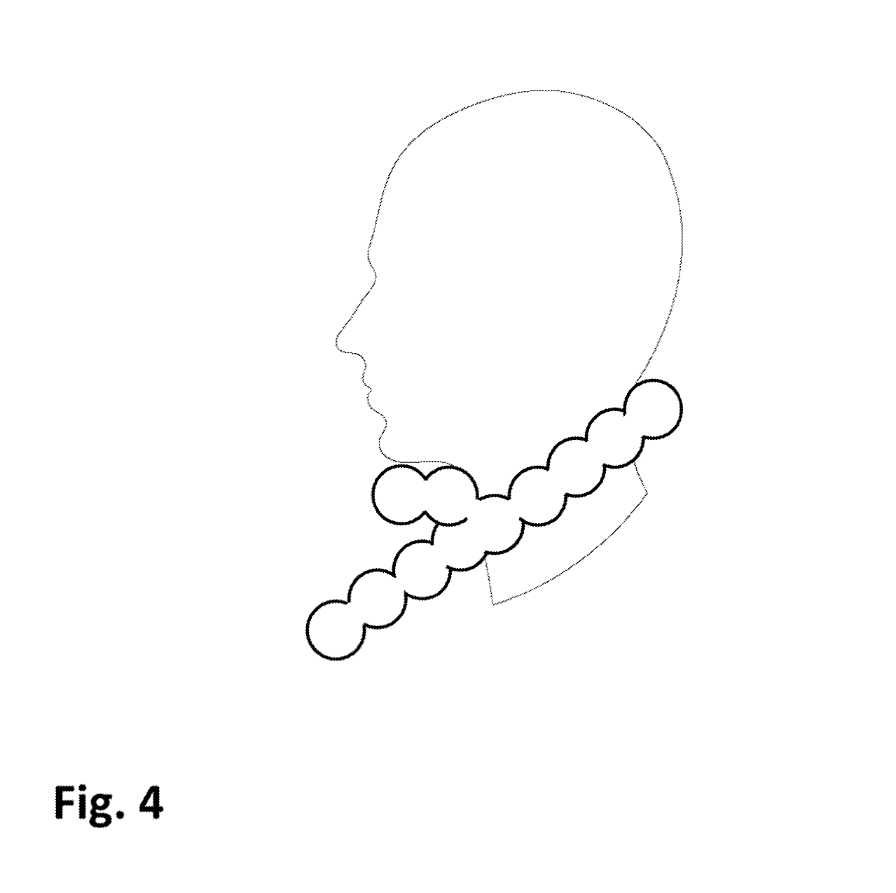

FIG. 4 is a diagram of one use case of the personal therapy device. A user's head is represented with the therapy device configured as a loop around the user's neck. The therapy device is acting like a neck support or brace. The therapy device is configured so two units 101 act as a chin support. The therapy device keeps it shape using the method shown in FIG. 3.

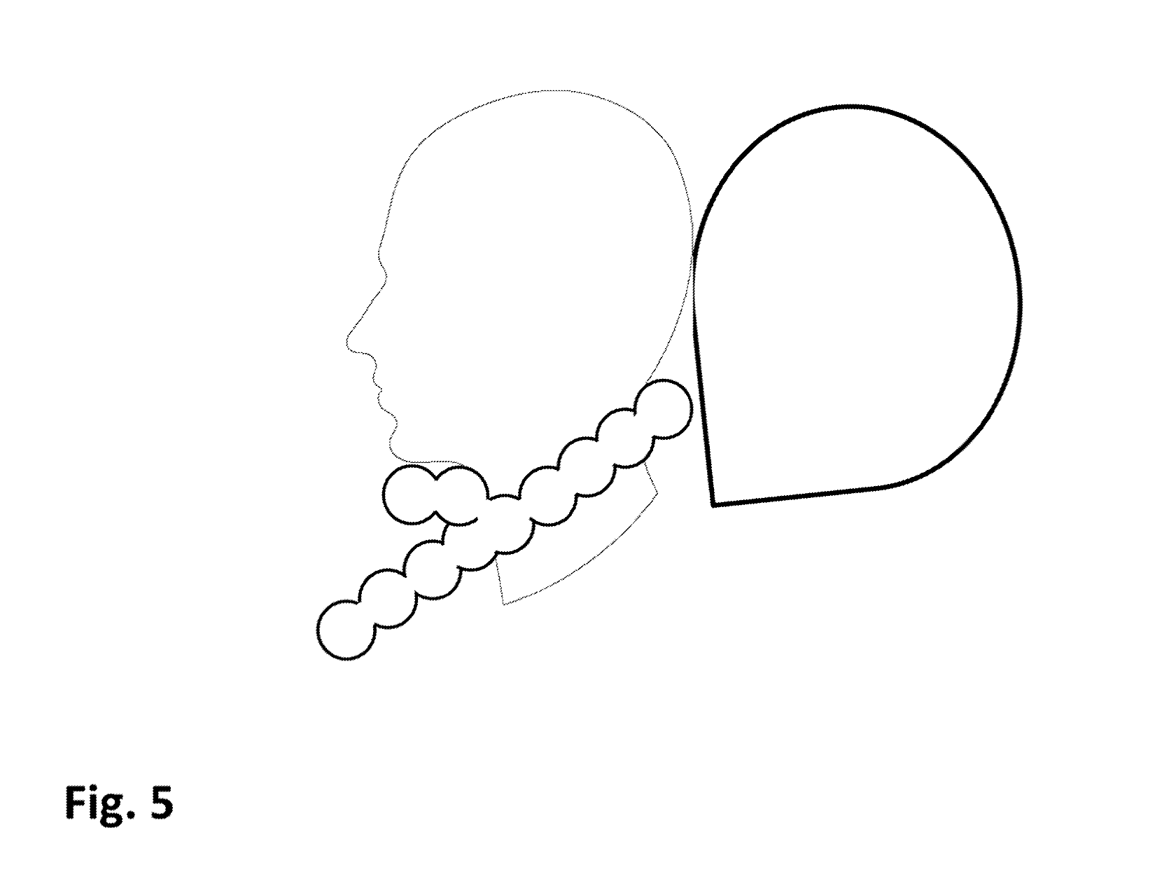

FIG. 5 shows a configuration of the therapy device 100 according to an embodiment. A user's head is represented with the therapy device configured as a loop around the user's neck. The user can lean their head in any direction to rest on the therapy device. A pillow 500 is shown to demonstrate how the therapy device can work in conjunction with a head rest to stabilize a user's neck and head.

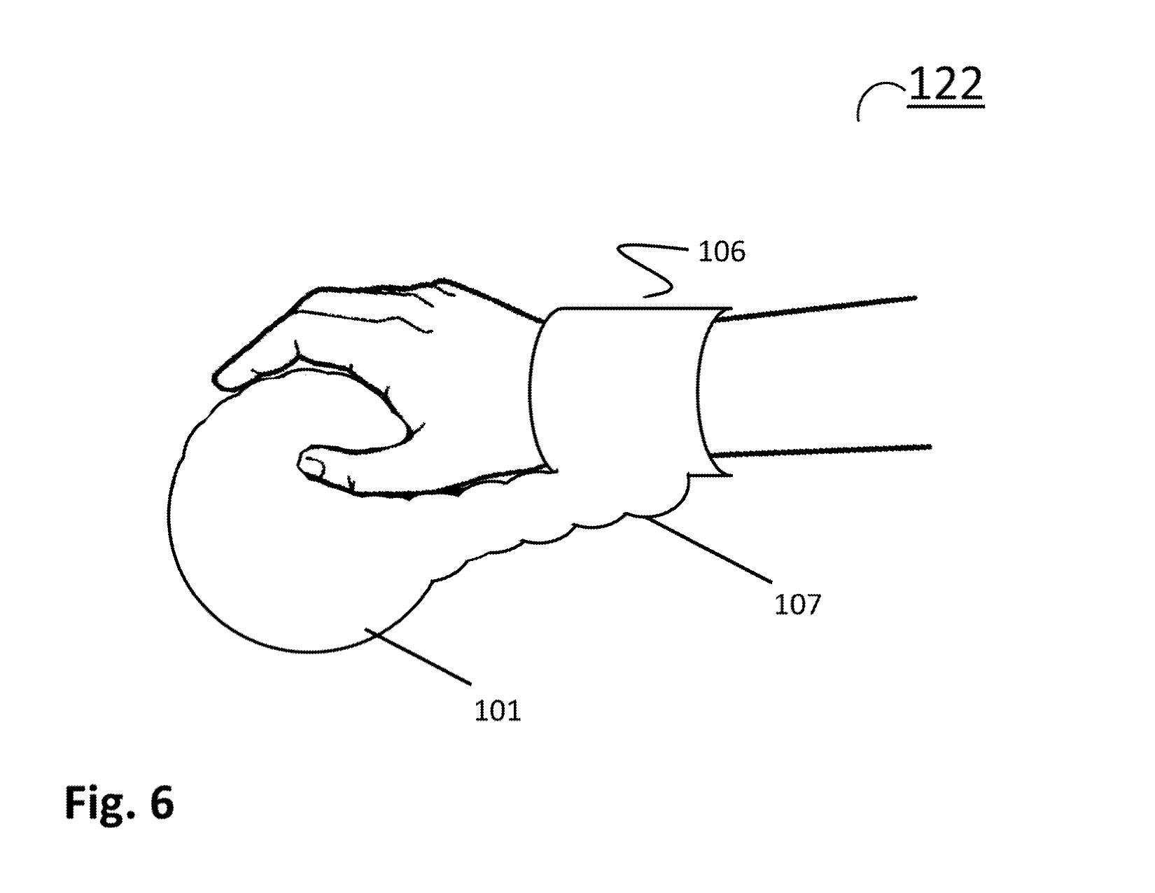

FIG. 6 shows a configuration of the therapy device according to an embodiment An expandable strap 106 is sized to fit over a person's hand and securely fit on the person's wrist, The configuration of device 122 makes it possible for the user to compress the deformable unit 101 with their hand for stress relief or as an exercise to strengthen hand muscles. In this embodiment unit 101 can be made of different densities or as a filled or hollow unit as described in FIG. 1C.

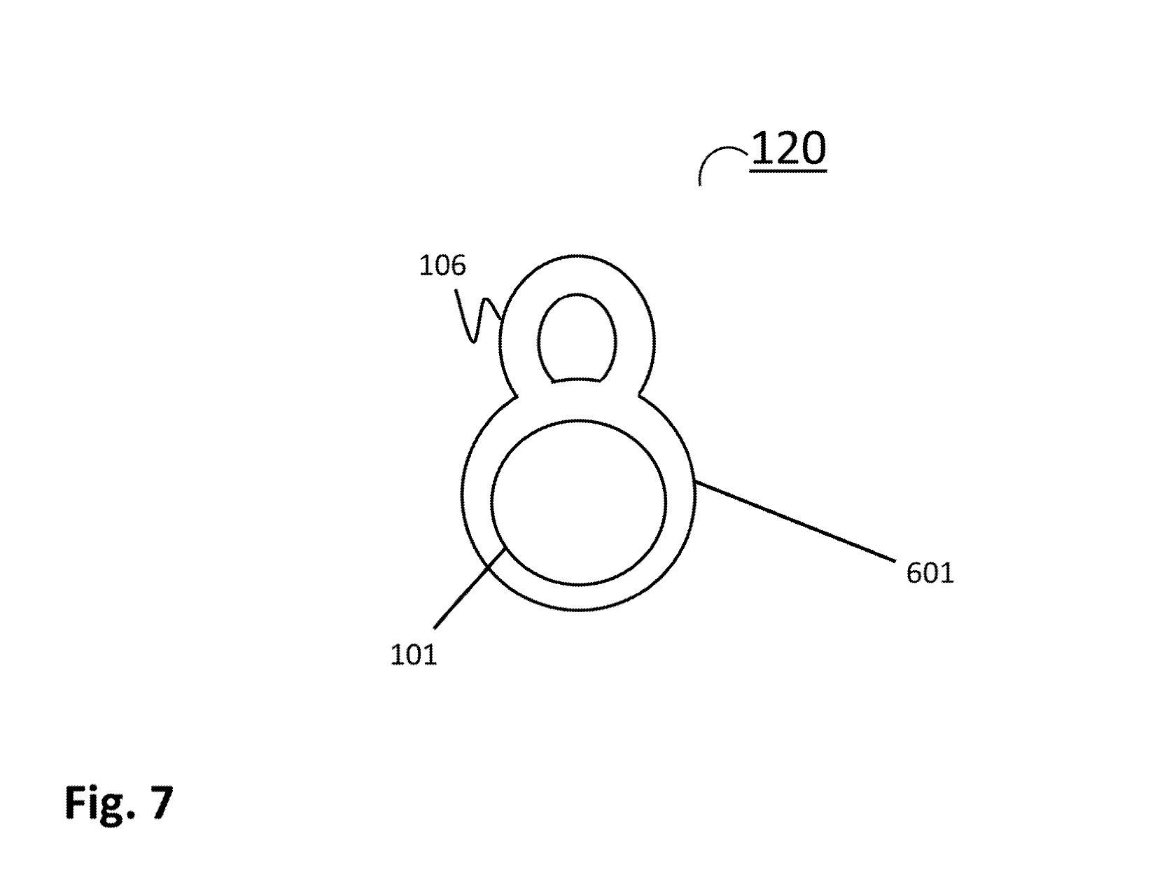

FIG. 7 is a diagram illustrating an embodiment of a personal therapy device 120 according to an embodiment. In this embodiment the therapy device contains a single unit 101. The single unit 101 is in an appropriately sized casing 601. The therapy device has an expandable strap 106 that can expand over a user's wrist or other body part.

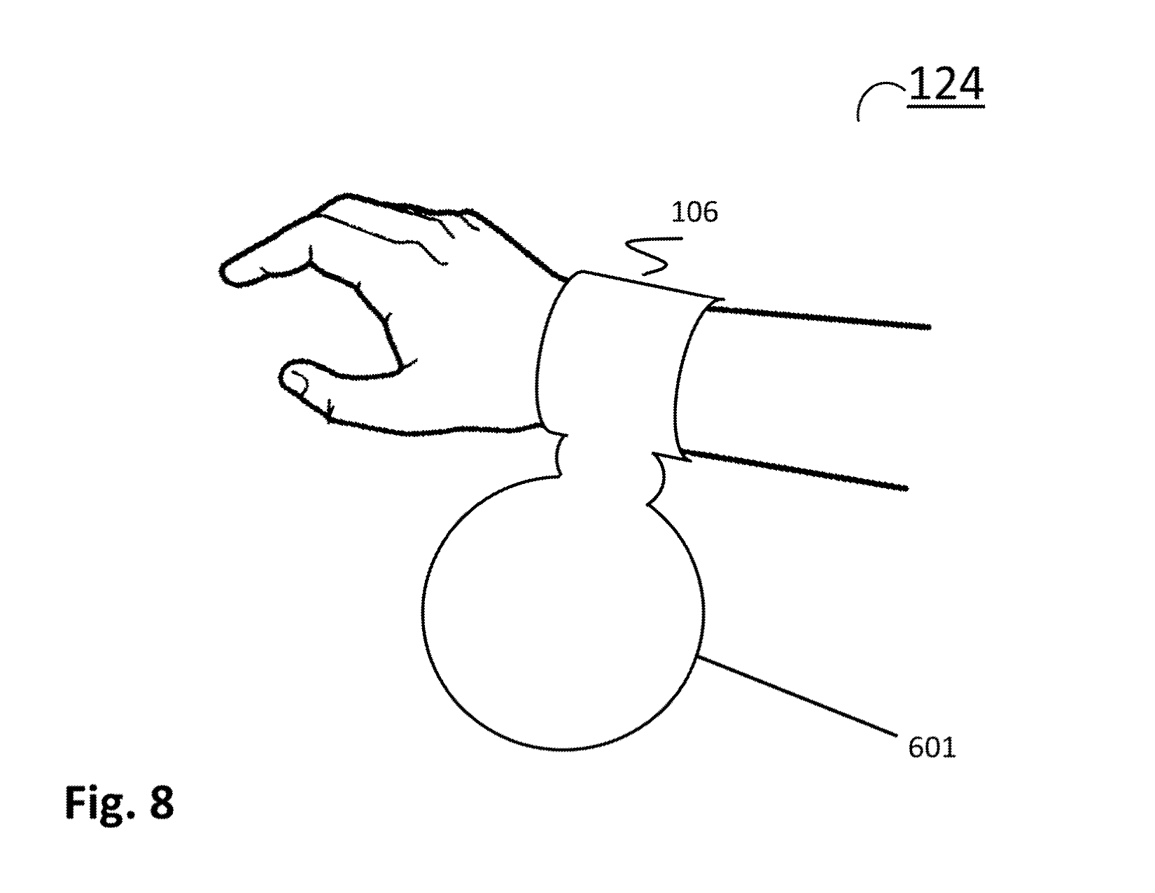

FIG. 8 shows a deployment 124 of the therapy device 120 according to an embodiment. The expandable strap 106 is slid over a user's wrist. The expandable strap 106 is pulled over the user's wrist and contracts to rest into position at the desired part of the user's wrist. The therapy device 120 is then used as a wrist or hand support.

What is claimed is:

1. A therapy device comprising;
a casing comprising a flexible material;
one or more individual units encased within the casing, comprising substantially spherical objects having a diameter in a range of 2.5 centimeters to 18 centimeters, wherein between each individual unit there is an inter-unit region that is variable in size; and
a loop fastener fixed to a terminal one of the individual units.

2. The therapy device of claim 1, wherein, the individual units are solid and comprised of a conformable material.

3. The therapy device of claim 1, wherein the individual units are hollow and have a wall thickness in a range of 0.20 centimeters to 1.0 centimeters.

4. The therapy device of claim 3, further comprising filler material for at least partially filling the individual units, wherein the filler material comprises polystryrene beads.

5. The therapy device of claim 1, wherein the therapy device is configurable to be secured into an adjustable loop shape using the loop fastener to encircle a chosen one of the inter-unit regions.

6. A therapy device comprising:
a casing comprising a flexible material;
one or more individual units encased within the casing, comprising compressible objects have a non-spherical shape and having a greatest dimension in a range of 15 centimeters to 18 centimeters. wherein between each individual unit there is an inter-unit region of the casing that is variable in size; and
a loop fastener fixed to a terminal one of the individual units.

7. A therapy device comprising:
a casing comprising a flexible material;
an individual unit encased within the casing, comprising a substantially spherical objects having a diameter in a range of 2.5 centimeters to 18 centimeters; and
an expandable strap fixed to a terminal the individual unit.

8. The therapy device of claim 7, wherein the expandable strap is configurable to secure the device to a human user's writs such that the user can grasp the individual unit.
